(19) United States
(12) Patent Application Publication
(10) Pub. No.: US 2015/0182359 A1
(43) Pub. Date: Jul. 2, 2015

(54) STENT AND METHOD OF USE

(71) Applicants: STRYKER CORPORATION, Kalamazoo, MI (US); STRYKER EUROPEAN HOLDINGS I, LLC, Kalamazoo, MI (US)

(72) Inventors: Brent C. Gerberding, San Jose, CA (US); Siddharth Loganathan, Santa Clara, CA (US)

(21) Appl. No.: 14/578,985

(22) Filed: Dec. 22, 2014

Related U.S. Application Data
(60) Provisional application No. 61/921,954, filed on Dec. 30, 2013.

(51) Int. Cl. A61F 2/89; A61F 2/95

(52) U.S. Cl. CPC A61F 2/89; A61F 2/95

(57) ABSTRACT

A stent having a compressed delivery configuration, an expanded configuration for implantation in a body lumen, a central body portion having a first plurality of struts, and an end portion having a second plurality of struts, wherein the first plurality of struts defines a central body pattern having a first axial stiffness, and the second plurality of struts defines an open cell configuration having a second axial stiffness greater than the first axial stiffness, the second axial stiffness sufficient to resist more than about 5% radial expansion of the end portion from the delivery configuration so long as at least about 20% of a length of the first end portion is radially constrained in the delivery configuration.

### RELATED APPLICATION DATA

The present application claims the benefit under 35 U.S.C. §119 to U.S. Provisional Application Ser. No. 61/921,954, filed Dec. 30, 2013. The foregoing application is hereby incorporated by reference into the present application in its entirety.

### FIELD

The present disclosure relates generally to medical devices and intravascular medical procedures and, more particularly, to stents and methods of using same.

### BACKGROUND

The use of intravascular medical devices has become an effective method for treating many types of vascular disease. In general, a suitable intravascular device is inserted into the vascular system of the patient and navigated through the vasculature to a desired target site. Using this method, virtually any target site in the patient's vascular system may be accessed, including the coronary, cerebral, and peripheral vasculature.

Medical devices such as stents, stent grafts, and vena cava filters, collectively referred to hereinafter as “stents,” are often utilized in combination with a delivery device for placement at a desired location within the body. A medical prosthesis, such as a stent for example, may be loaded onto a stent delivery device and then introduced into the lumen of a body vessel in a delivery configuration having a reduced diameter. Once delivered to a target location within the body, the stent may then expand or be expanded to an expanded configuration within the vessel to support and reinforce the vessel wall while maintaining the vessel in an open, unobstructed condition.

Stents are generally tubular devices for insertion into body lumens. However, it should be noted that stents may be provided in a wide variety of sizes and shapes. Balloon expandable stents require mounting over a balloon, positioning, and inflation of the balloon to expand the stent radially outward. Self-expanding stents expand into place when unconstrained, without requiring assistance from a balloon. A self-expanding stent may be biased so as to expand upon release from the delivery catheter and/or include a shape-memory component which allows the stent to expand upon exposure to a predetermined condition. Self-expanding stents are biased to an expanded configuration. Some stents may be characterized as hybrid stents which have some characteristics of both self-expandable and balloon expandable stents.

Stents may be constructed from a variety of materials such as stainless steel, Elgiloy, nickel, titanium, nitinol, shape memory polymers, etc. Stents may also be formed in a variety of manners as well. For example a stent may be formed by etching or cutting the stent pattern from a tube or sheet of stent material; a sheet of stent material may be cut or etched according to a desired stent pattern whereupon the sheet may be rolled or otherwise formed into the desired substantially tubular, bifurcated or other shape of the stent; one or more wires or ribbons of stent material may be woven, braided or otherwise formed into a desired shape and pattern. The density of the braid in braided stents is measured in picks per inch. Stents may include components that are welded, bonded or otherwise engaged to one another.

Typically, a stent is implanted in a blood vessel or other body lumen at the site of a stenosis or aneurysm by so-called “minimally invasive techniques” in which the stent is compressed radially inwards and is delivered by a catheter to the site where it is required through the patient's skin or by a “cut down” technique in which the blood vessel concerned is exposed by minor surgical means. When the stent is positioned at the correct location, the stent is caused or allowed to expand to a predetermined diameter in the vessel. Many delivery devices include sheaths or catheters, and delivery members having bumpers thereon to push and pull stents through the sheaths and catheters. A catheter may be bent while navigating through torturous vasculature.

Some stents are deployed by loading them proximally from an introducer sheath into a pre-positioned microcatheter. The stent is then pushed through the microcatheter for approximately 150 cm until it is deployed from the distal end of the catheter at the treatment site.

This “empty catheter” technique is different from the more traditional self-expanding stent delivery technique, which includes pre-loading the stent adjacent the distal end of the catheter and then simultaneously tracking the stent and catheter to the treatment site. The evolution of the empty catheter technique was driven by the extremely tortuous anatomy commonly found in the intracranial circulation.

Open cell stents are more flexible because they have free apices that are not connected to other struts of the stent. Increase flexibility facilitates deployment of stents into smaller lumens, like those in the neuro-vasculature. However, when stents have open cells at their proximal and distal ends, those ends can flare when they are unsheathed (unconstrained). If an open cell stent is unsheathed or unconstrained, even temporarily, during the delivery process, but before being delivered to a target cite, the flared proximal and distal ends can be damaged and/or interfere with delivery.

For instance, FIG. 1 depicts a prior art self-expanding stent 10 with open cell proximal and distal ends 12, 14 being transferred from a storage sheath 16 to a catheter 18, during an empty catheter technique stent delivery, as described above. Stents 10 are typically stored in sheaths 16 to facilitate handling while minimizing damage. In the empty catheter technique described above, stents 10 are loaded from sheaths 16 into catheters 18 for delivery. The proximal end 20 of the catheter 18 is connected to a hub 22, which defines a proximally-opening funnel 24 to facilitate insertion of the distal end 26 of the storage sheath 16 into the proximal end 56 of the hub 22 (and catheter 18). The stent delivery system also includes a guidewire 30 carrying a proximal bumper 32, which is disposed adjacent the proximal end 12 of the stent 10 and configured to push the stent 10 distally through the catheter 18.

As shown in FIGS. 1 and 2, a space 36 is formed at the interface of the sheath 16 and the funnel 24 because the distal end 26 of the storage sheath 16 does not fit perfectly into the funnel 24 (i.e., the tolerance of the fit). This space 36 spans an open distance 50 along the longitudinal axis of the stent delivery system. This imperfect fit is exacerbated by the lack of uniformity in catheters 18 and hubs 22, especially between different manufacturers. Accordingly, a single storage sheath 16 must be able to deliver stents 10 into a variety of catheters 18 with different proximal hubs 22, which define funnels 24 and other receptacles with different shapes. As a result, storage sheaths 16 will not fit perfectly into all funnels 24/hubs 22 and spaces 36 will be formed at the interface of sheaths 16 and hubs 22.

One consequence of the presence of spaces 36 at the interface of sheaths 16 and hubs 22 is that the proximal end 12 of open cell stents 10 can flare open upon exiting their restraining sheaths 16, as shown in FIG. 2. When the proximal end 12 of the open cell stent 10 fully exits the sheath 16 into the cone-shaped space 36, the proximal end 12 of the stent 10 is no longer constrained and opens into the cone-shaped space 36 like an umbrella. After the proximal end 12 of the stent 10 opens, distally directed delivery force on the guidewire 30 can push the proximal bumper 32 into the opened proximal end 12. As a result, the proximal bumper 32 loses efficiency in pushing the stent 10 in a distal direction and the proximal bumper 32 may jam in the distal (smaller) end 28 of the hub 22/funnel 24, causing the stent delivery procedure to fail. Movement of the proximal bumper 32 into a partially opened end 12, 14 of a stent 10 before the completion of delivery is called “corking.”

FIGS. 3-5 depict another stent delivery system for delivering a prior art stent 10 having open cell proximal and distal ends 12, 14. The stent delivery system includes proximal and distal bumper 32, 34 carried on a guidewire 30, and disposed adjacent the proximal and distal ends 12, 14 of the prior art stent 10. The proximal and distal bumpers 32, 34 are respectively configured to move the stent 10 in distal and proximal directions. The proximal bumper 32 can cork into the proximal end 12 of the stent 10 when the unconstrained proximal end 12 flairs open in the space 36 between the distal end 26 of the sheath 16 and the proximal end 20 of the catheter 18. The corking of the proximal bumper 32 is similar to the corking shown in FIG. 2.

FIG. 3 depicts an early stage of the delivery process in which the proximal and distal bumpers 32, 34 and the stent 10 therebetween are all contained within the sheath 16. When the distal bumper 34 and the distal end 14 of the stent 10 are pushed distally out of the sheath 16 and into the space 36, the unconstrained distal end 14 flares open, as shown in FIG. 4. While the distal end 14 of the stent 10 may be forced to close as the stent 10 is pushed into the distal end 28 of the hub 22/funnel 24, friction between the distal end 14 of the stent 10 and the hub 22 may damage the distal end 14 of the stent 10. Further, any attempt to withdraw the stent 10 into the sheath 16 may result in corking of the distal bumper 34 into the open distal end 14 of the stent 10, as shown in FIG. 5.

Accordingly, there exists a need for open cell stents that do not flare open when partially unsheathed. SUMMARY

In one embodiment of the disclosed inventions, a stent has a delivery configuration sized for introduction into a body lumen, and an expanded configuration for implantation in the body lumen, into which the stent is biased. The stent includes a central body portion having a first plurality of struts and an end portion having a second plurality of struts. The first plurality of struts defines a central body pattern having a first axial stiffness. The second plurality of struts defines an open cell configuration having a second axial stiffness greater than the first axial stiffness. The second axial stiffness is sufficient to resist more than about 5% radial expansion of the end portion from the delivery configuration, so long as at least about 20% of a length of the first end portion is radially constrained in the delivery configuration.

In some embodiments, the open cell configuration includes a plurality of circumferential rings formed from the second plurality of struts, and a plurality of connectors coupling respective pairs of the plurality of circumferential rings. The open cell configuration may also include a marker coupled to one of the plurality of circumferential rings by a connector. One of the circumferential rings may include struts forming a zig-zag pattern.

In some embodiments, when the stent is in the delivery configuration, two connectors of the plurality of connectors are circumferentially aligned with two struts in respective circumferential rings, and the two struts are coupled to each other by one of the two connectors. When the stent is in the delivery configuration, the two connectors and the two struts may also be circumferentially aligned with a third strut, where the third strut forms part of a marker, and where the third strut is coupled to one of the two struts by a second one of the two connectors.

In another embodiment of the disclosed inventions, a stent includes a central body portion including a first plurality of struts, an open cell first end portion including a second plurality of struts coupled to a first end of the central body portion, and an open cell second end portion including a third plurality of struts coupled to a second end of the central body portion. The first and second end portions have respective first and second lengths. The open cell first and second end portions have respective first and second axial stiffnesses sufficient to resist more than about 5% radial expansion of the respective first and second end portions from the delivery configuration, so long as at least about 20% of the respective first and second lengths of the respective first and second end portions is radially constrained in the delivery configuration.

In various embodiments, the first and second end portions each include a plurality of circumferential rings formed from the respective second and third plurality of struts, and a plurality of connectors coupling respective pairs of the plurality of circumferential rings. When the stent is in the delivery configuration, two connectors of the plurality of connectors are circumferentially aligned with two struts in respective circumferential rings of the plurality, and the two struts are coupled to each other by one of the two connectors. The first and second lengths may be approximately equal or different. The first and second axial stiffnesses may be approximately equal or different.

In yet another embodiment of the disclosed inventions, a stent delivery system, includes a delivery catheter having a delivery lumen in communication with an open proximal end, the delivery catheter having a proximal hub surrounding the open proximal end, a storage sheath having an open distal end, where the delivery catheter hub is sized to receive a distal end portion of the storage sheath such that the open proximal end of the delivery catheter is spaced apart from the open distal end of the storage sheath by an open distance, and a stent disposed in the storage sheath. The stent has a delivery configuration sized for introduction into the body lumen through the delivery catheter, and an expanded configuration for implantation in the body lumen, into which the stent is biased. The stent includes a central body portion including a first plurality of struts, and an open cell end portion including a second plurality of struts. The end portion has an axial stiffness such that the stent can be transferred out the open distal end of the storage sheath into the open proximal end of the delivery catheter without the end portion substantially expanding from the delivery configuration, so long as the end portion has a longitudinal length about 25% greater than a longitudinal length of the space defined by the storage sheath and the proximal hub.

In some embodiments, the open cell end portion is a first open cell end portion, and the axial stiffness is a first axial stiffness. In such embodiments, the stent may also include a second open cell end portion including a third plurality of struts. The second end portion has a second axial stiffness such that the stent can be transferred out the open distal end of the storage sheath into the open proximal end of the delivery catheter without the second end portion substantially expanding from the delivery configuration, so long as the second end portion has a longitudinal length about 25% greater than a longitudinal length of the space defined by the storage sheath and the proximal hub. The first and second axial stiffnesses may be approximately equal or different.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of its scope.

FIGS. 1-5 are detailed cross-sectional views of stent delivery systems delivering a prior art stent using the empty catheter technique.

FIGS. 6 and 7 are detailed top views of stents according to various embodiments of the disclosed inventions that have been cut open along their lengths and unrolled into flat sheets, with select components of the stents omitted for clarity. FIGS. 6 and 7 depict stents in a radially collapsed “delivery” configuration. While FIGS. 6 and 7 omit select elements of the stents depicted therein, one of skill in the art will recognize that FIGS. 6 and 7 depict patterns that can be repeated in the axial and circumferential directions to form stents of any size.

FIGS. 8 and 9 are detailed cross-sectional views of stent delivery systems delivering a stent according to various embodiments of the disclosed inventions using the empty catheter technique.

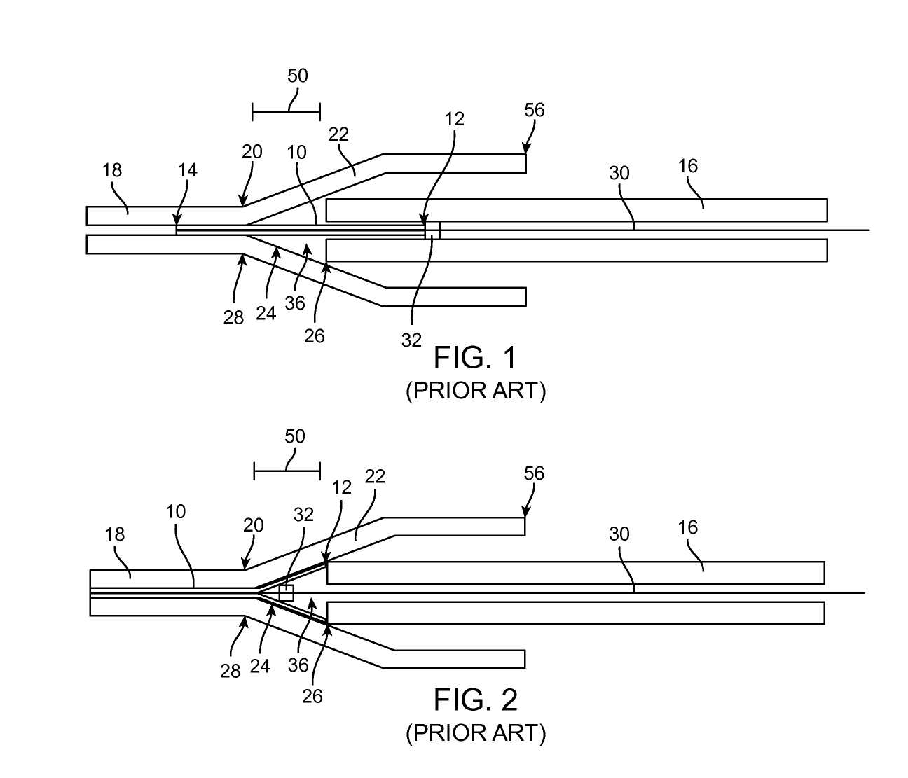

### DETAILED DESCRIPTION

For the following defined terms, these definitions shall be applied, unless a different definition is given in the claims or elsewhere in this specification.

All numeric values are herein assumed to be modified by the terms “about” or “approximately,” whether or not explicitly indicated. The terms “about” and “approximately” generally refers to a range of numbers that one of skill in the art would consider equivalent to the recited value (i.e., having the same function or result). In many instances, he terms “about” and “approximately” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, an “open cell stent” is a stent having an apex that is not connected to any other stent element. As used in this specification and the appended claims, a stent “substantially expands” when it undergoes more than about 5% radial expansion (i.e., increase in cross-sectional radius). As used in this specification and the appended claims, the term “circumferentially aligned” means that two lines are within approximately ±5 degrees of being parallel each other.

Various embodiments of the disclosed inventions are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated.

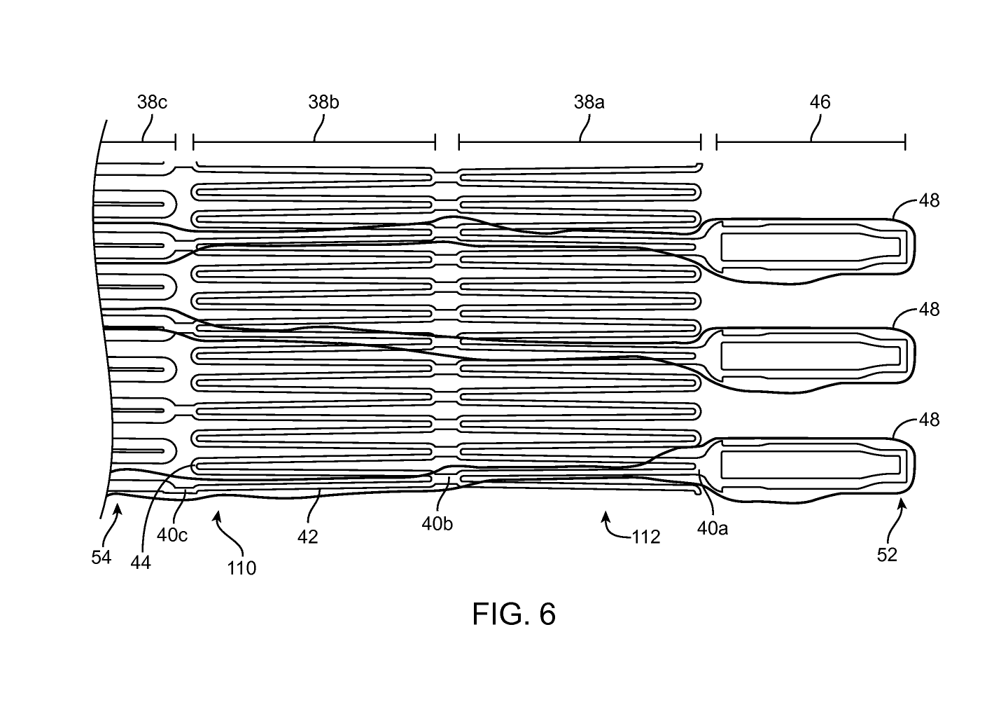

FIG. 6 depicts a portion of a stent 110 provided in accordance with one embodiment of the disclosed inventions, wherein the stent 110 is shown having been cut open along its length and unrolled into a flat sheet. While only a portion of the stent 110 is depicted in FIG. 6, the pattern shown in FIG. 6 is repeated along the circumference of the stent 110. The configuration of the various parts of the stent 110 depicted in FIG. 6 represents the configuration found in a stent 110 that is in a radially collapsed configuration, which is the delivery configuration. The stent 110 is formed in a single layer between a proximal end 112 and a distal end 114. The stent 110 also includes a central body portion. In some embodiments, the stent 110 is etched or cut (e.g., by laser) from a solid tube comprised of metals, polymers, composites and other materials, such as PET, PTFE, stainless steel, Elgiloy, nickel, titanium, Nitinol, shape memory polymers, and other biocompatible materials. The stent 110 may also be cut from a flat sheet and welded at a seam (e.g., spot-welded). The stent 110 may also be vapor deposited (e.g., Nitinol) on a mandrel that is machined with the desired pattern. The stent 110 can have a molded or other non-wire construction. Moreover, various parts of the stent 110 (i.e., “stent elements”) can be made as separate parts, then joined together (e.g., by welding or adhesives).

The stent 110 shown in FIG. 6 includes a plurality of rings 38 of stent elements connected to each other by ring connectors 40. When the stent 110 is cut open and unrolled, the rings 38 form rows 38 of stent elements. In the depicted stent 110, each ring 38 depicted in FIG. 6 includes a continuous zig-zag shape along a circumferential direction of the stent 110. Each zig-zag shape is formed by a plurality of elongate segments 42 joined at vertices 44. FIG. 6 depicts first, second, and third rings 38a, 38b, 38c and a plurality of markers 46. The first ring 38a is the proximal most of ring on the stent 110. Some of the proximal vertices 44 of the first ring 38a are connected to markers 46 by first ring connectors 40a. Other proximal vertices 44 of the first ring 38a are not connected to any other stent element, thereby forming open cells at the proximal end 112 of the stent 110.

The second ring 38b is immediately distal of the first ring 38a and connected to the first ring 38a by a plurality of second ring connectors 40b. The first and second rings 38a, 38b are identical and each distal vertex 44 of the first ring 38a is connected to a corresponding proximal vertex 44 of the second ring 38b by a second ring connector 40b. Some of the proximal vertices 44 of the second ring 38b are not connected to any other stent element, thereby forming additional open cells at the proximal end 112 of the stent 110.

The third ring 38c is immediately distal of the second ring 38b and connected to the second ring 38b by a plurality of third ring connectors 40c. The third ring 38c has wider elongate segments 42 than the first and second rings 38a, 38b. Every other proximal vertex 44 of the third ring 38c is connected to a distal vertex 44 of the second ring 38b by a third ring connector 40c. Every third distal vertex 44 of the second ring 38b (i.e., vertices 1, 4, 7, 10, 13, etc.) is connected to the third ring 38c, such that such “connected” vertices 44 of the second ring 38b are separated by two “unconnected” vertices 44 of the second ring.

In the illustrated embodiment, with the stent 110 in its radially collapsed configuration, each of the first ring connectors 40a (connecting markers 46 to vertices 44 of the first ring 38a) depicted in FIG. 6 are circumferentially aligned with respective second and third ring connectors 40b, 40c (connecting vertices 44 of the second ring 38b to vertices 44 of the first ring 38a and third ring 38c, respectively). With respect to FIG. 6, the term “circumferentially aligned” means that the line between the first and second ring connectors 40a, 40b is within approximately ±5 degrees of the line between the second and third ring connectors 40b, 40c. The aligned first, second, and third ring connectors 40a, 40b, 40c, together with respective elongate segments 42 from the first, second, and third rings 38a, 38b, 38c, and the marker 46 form virtual struts 48 that span all three rings 38a, 38b, 38c and the marker 46. A virtual strut 48 is a stent segment formed by aligned first, second, and third ring connectors 40a, 40b, 40c, elongate segments 42 from the first, second, and third rings 38a, 38b, 38c, and the marker 46.

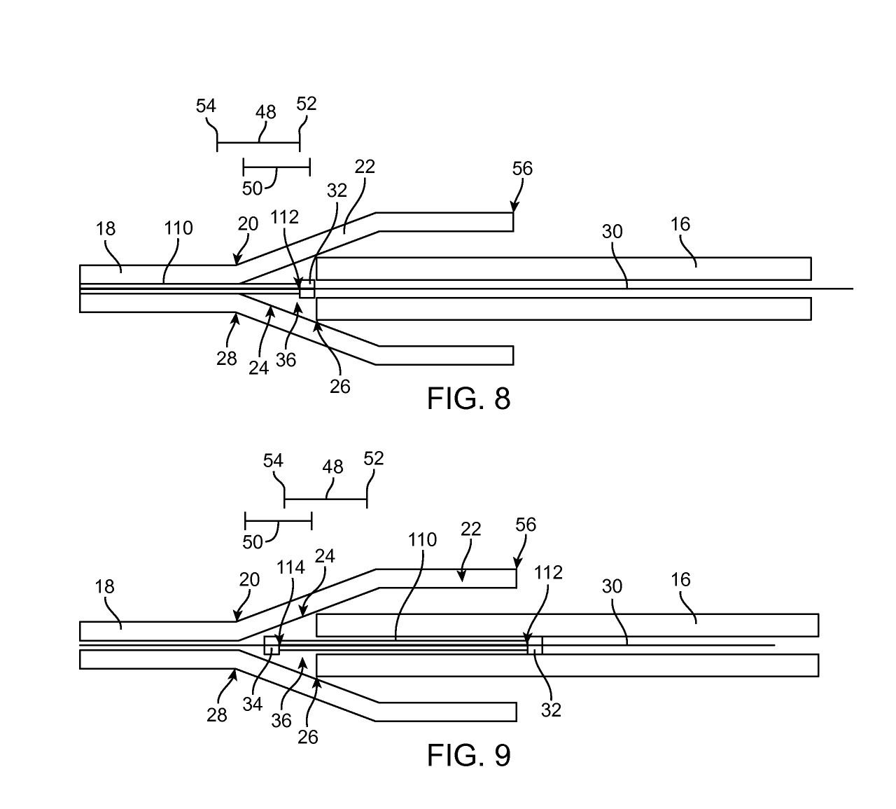

The stent 110 is configured such that each virtual strut 48 is longer than the open distance 50 between the distal end 26 of the sheath 16 and the proximal end 20 of the catheter 18 (see FIGS. 8 and 9 for the open distance 50). In other words, the virtual strut 48 spans, and is longer than, the open distance 50. In fact, each virtual strut 48 is at least 25% longer than the open distance 50 between the distal end 26 of the sheath 16 and the proximal end 20 of the catheter 18. As shown in FIG. 8, the virtual strut 48 will resist flaring of the proximal end 112 of the stent 110 in the open distance 50, because the distal end 52 of the virtual strut 48 at the proximal end 112 of the stent 110 will enter the proximal end 20 of the catheter 18 before the proximal end 54 of the virtual strut 48 exits the distal end 26 of the sheath 16. With a sufficient number of virtual struts 48 (e.g., one at every marker 46) distributed along the circumference of the stent 110, the virtual struts 48 will minimize flaring at the proximal end 112 of an open cell stent 110, therefore allowing the proximal bumper 32 to push the stent 110 distally from the sheath 16 into the hub 22 and the catheter 18 with no corking of the proximal bumper 32.

As a result of the presence of the virtual struts 48, the proximal end 112 of the stent 110 is stiffer than a central body portion of the stent 110. Further, because the virtual strut 48 is at least 25% longer than the open distance 50, the stent 110 will not significantly expand (i.e., not more than 5% radial expansion) as long as at least 20% of the virtual strut 48 at the proximal end 112 of the stent 110 is radially constrained by the catheter 18.

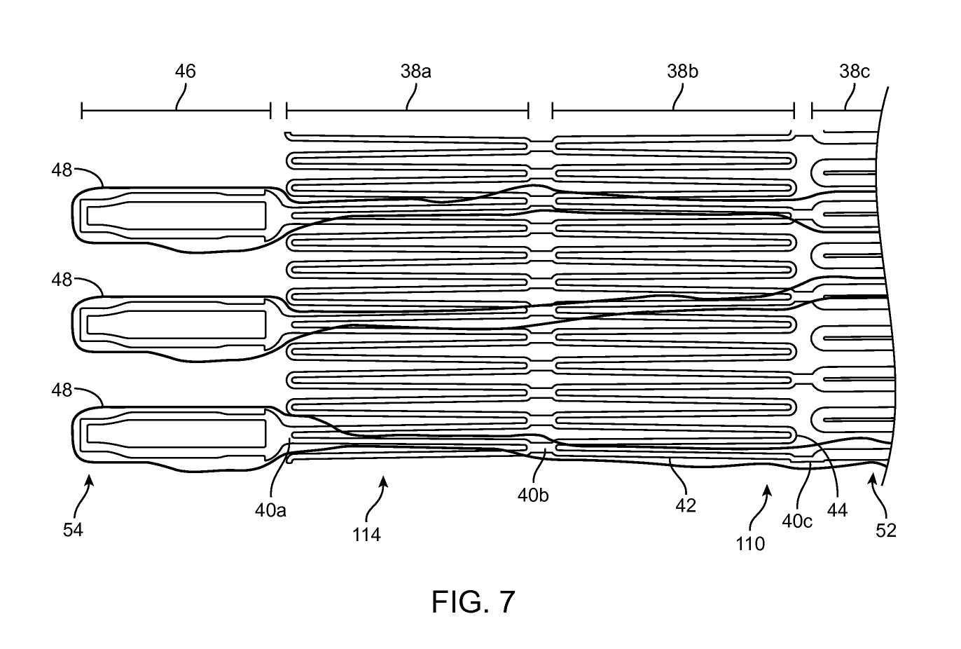

FIG. 7 depicts a distal end 114 of an open cell stent 110 with a construction that is the mirror image of the stent proximal end 112 depicted in FIG. 6. In fact, the proximal and distal ends 112, 114 depicted in FIGS. 6 and 7 can belong to the same open cell stent 110. Like the proximal end 112 depicted in FIG. 6, the distal end 114 is depicted in FIG. 7 with the stent 110 in its radially collapsed configuration. The stent 110 includes first, second, and third rings 38a, 38b, 38c and a plurality of markers 46. These stent elements are serially connected to each other by first, second, and third ring connectors 40a, 40b, 40c, like in the stent proximal end 112 depicted in FIG. 6.

Each of the first ring connectors 40a in the distal end 114 depicted in FIG. 7 are approximately circumferentially aligned with respective second and third ring connectors 40b, 40c. The aligned first, second, and third ring connectors 40a, 40b, 40c, together with respective elongate segments 42 from the first, second, and third rings 38a, 38b, 38c, and the marker 46 form virtual struts 48 that spans all three rings 38a, 38b, 38c and the marker 46.

The stent 110 is configured such that each virtual strut 48 is longer than the open distance 50 between the distal end 26 of the sheath 16 and the proximal end 20 of the catheter 18, i.e., the virtual strut 48 spans, and is longer than, the open distance 50. In fact, each virtual strut 48 is at least 25% longer than the open distance 50 between the distal end 26 of the sheath 16 and the proximal end 20 of the catheter 18. As shown in FIG. 9, the distal end 52 of the virtual strut 48 will enter the proximal end 20 of the catheter 18 before the proximal end 54 of the virtual strut 48 exits the distal end 26 of the sheath 16, thereby minimizing flaring at the distal end 114 of an open cell stent 110. This, in turn, minimizes damage to the distal end 114 of the stent 110 and allows a distal bumper 34 to withdraw a partially deployed stent 110 proximally from the hub 22 and the catheter 18 into the sheath 16, with no corking of the distal bumper 34 into the distal end 114 of the stent 110.

As a result of the presence of the virtual struts 48, the distal end 114 of the stent 110 is stiffer than a central body portion of the stent 110. Further, because the virtual strut 48 is at least 25% longer than the open distance 50, the stent 110 will not significantly expand (i.e., not more than 5% radial expansion) as long as at least 20% of the virtual strut 48 at the distal end 114 of the stent 110 is radially constrained by the sheath 16.

Stents 110 made from radiopaque materials do not require markers 46 for visualization. However, such stents 110 may include proximal and distal end protrusions to minimize damage to the proximal and distal ends 112, 114 of the stent 110 while the stent 110 interacts with respective proximal and distal bumpers 32, 34. The virtual struts 48 (proximal and distal) in such stents 110 end at the proximal and distal ends 112, 114 of the stent 110, which may, or may not, include protrusions.

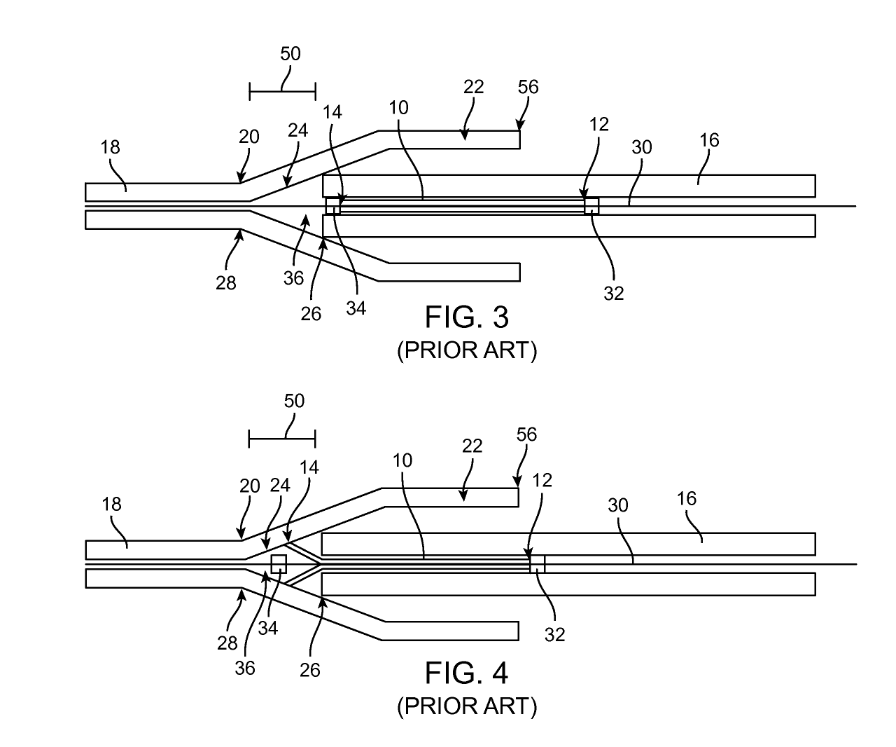

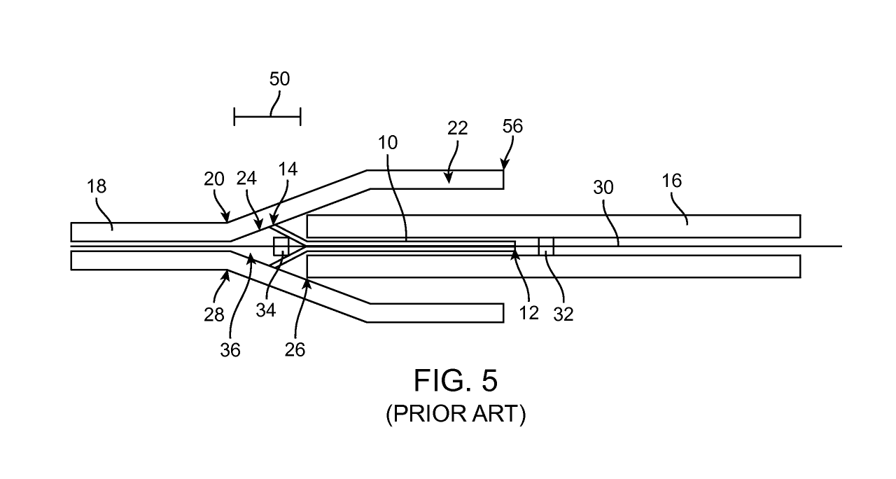

The stent elements depicted in the illustrated embodiments are exemplary and not limiting. The scope of the claims encompasses stents 110 that have virtual struts 48, which resist flaring of the stent 110 into open spaces before the stent 110 is delivered into a target site. While the elongated segments 42 of the rings 38a, 38b, 38c depicted in FIGS. 6 and 7 have substantially the same width, the elongated segments 42 along the virtual struts 48 can have greater widths to increase the resistance to flaring of the proximal and distal ends 112, 114 of the stent 110. While the distal bumper 34 illustrated in FIGS. 3-5 and 9 is disposed adjacent and distal to the distal end 114 of the stent 110, the distal bumper 34 may be located under the distal end 114 of the stent 110. Even in such embodiments, resisting flaring of the distal end 114 of the stent 110 with virtual struts 48 will increase the effectiveness of the distal bumper 34 in withdrawing stents 110 into sheaths 16.

Although particular embodiments of the disclosed inventions have been shown and described herein, it will be understood by those skilled in the art that they are not intended to limit the present inventions, and it will be obvious to those skilled in the art that various changes and modifications may be made (e.g., the dimensions of various parts) without departing from the scope of the disclosed inventions, which is to be defined only by the following claims and their equivalents. The specification and drawings are, accordingly, to be regarded in an illustrative rather than restrictive sense. The various embodiments of the disclosed inventions shown and described herein are intended to cover alternatives, modifications, and equivalents of the disclosed inventions, which may be included within the scope of the appended claims.

What is claimed is:

1. A stent having a delivery configuration sized for introduction into a body lumen, and an expanded configuration for implantation in the body lumen, the stent biased to the expanded configuration, the stent comprising:
a central body portion comprising a first plurality of struts, the first plurality of struts defining a central body pattern having a first axial stiffness; and
an end portion comprising a second plurality of struts, the second plurality of struts defining an open cell configuration having a second axial stiffness greater than the first axial stiffness,
the second axial stiffness sufficient to resist more than about 5% radial expansion of the end portion from the delivery configuration, so long as at least about 20% of a length of the first end portion is radially constrained in the delivery configuration.

2. The stent of claim 1, the open cell configuration comprising:
a plurality of circumferential rings formed from the second plurality of struts; and
a plurality of connectors coupling respective pairs of the plurality of circumferential rings.

3. The stent of claim 2, the open cell configuration further comprising:
a marker coupled to one of the plurality of circumferential rings by a connector.

4. The stent of claim 2, wherein one of the circumferential rings comprises struts forming a zig-zag pattern.

5. The stent of claim 2, wherein, when the stent is in the delivery configuration, two connectors of the plurality of connectors are circumferentially aligned with two struts in respective circumferential rings, and
wherein the two struts are coupled to each other by one of the two connectors.

6. The stent of claim 5, wherein, when the stent is in the delivery configuration, the two connectors and the two struts are circumferentially aligned with a third strut,
wherein the third strut forms part of a marker, and
wherein the third strut is coupled to one of the two struts by a second one of the two connectors.

7. A stent having a delivery configuration sized for introduction into a body lumen, and an expanded configuration for implantation in the body lumen, the stent biased to the expanded configuration, the stent comprising:
a central body portion comprising a first plurality of struts;
an open cell first end portion comprising a second plurality of struts coupled to a first end of the central body portion, the first end portion having a first length; and
an open cell second end portion comprising a third plurality of struts coupled to a second end of the central body portion, the second end portion having a second length,
the open cell first and second end portions have respective first and second axial stiffnesses sufficient to resist more than about 5% radial expansion of the respective first and second end portions from the delivery configuration, so long as at least about 20% of the respective first and second lengths of the respective first and second end portions is radially constrained in the delivery configuration.

8. The stent of claim 7, the first end portion comprising:
a plurality of circumferential rings formed from the second plurality of struts; and
a plurality of connectors coupling respective pairs of the plurality of circumferential rings.

9. The stent of claim 8, wherein, when the stent is in the delivery configuration, two connectors of the plurality of connectors are circumferentially aligned with two struts in respective circumferential rings of the plurality, and
wherein the two struts are coupled to each other by one of the two connectors.

10. The stent of claim 7, the second end portion comprising:
a plurality of circumferential rings formed from the third plurality of struts; and
a plurality of connectors coupling respective pairs of the plurality of circumferential rings.

11. The stent of claim 10, wherein, when the stent is in the delivery configuration, two connectors of the plurality of connectors are circumferentially aligned with two struts in respective circumferential rings of the plurality, and
wherein the two struts are coupled to each other by one of the two connectors.

12. The stent of claim 7, wherein the first and second lengths are approximately equal.

13. The stent of claim 7, wherein the first and second lengths are different.

14. The stent of claim 7, wherein the first and second axial stiffnesses are approximately equal.

15. The stent of claim 7, wherein the first and second axial stiffnesses are different.

16. A stent delivery system, comprising:
a delivery catheter having a delivery lumen in communication with an open proximal end, the delivery catheter having a proximal hub surrounding the open proximal end;
a storage sheath having an open distal end, wherein the delivery catheter hub is sized to receive a distal end portion of the storage sheath such that the open proximal end of the delivery catheter is spaced apart from the open distal end of the storage sheath by an open distance; and
a stent disposed in the storage sheath, the stent having a delivery configuration sized for introduction into the body lumen through the delivery catheter, and an expanded configuration for implantation in the body lumen, the stent biased to the expanded configuration, the stent comprising
a central body portion comprising a first plurality of struts; and
an open cell end portion comprising a second plurality of struts, the end portion having an axial stiffness such that the stent can be transferred out the open distal end of the storage sheath into the open proximal end of the delivery catheter without the end portion substantially expanding from the delivery configuration, so long as the end portion has a longitudinal length about 25% greater than a longitudinal length of the space defined by the storage sheath and the proximal hub.

17. The system of claim 16, wherein the open cell end portion is a first open cell end portion, and wherein the axial stiffness is a first axial stiffness, the stent further comprising:
a second open cell end portion comprising a third plurality of struts, the second end portion having a second axial stiffness such that the stent can be transferred out the open distal end of the storage sheath into the open proximal end of the delivery catheter without the second end portion substantially expanding from the delivery configuration, so long as the second end portion has a longitudinal length about 25% greater than a longitudinal length of the space defined by the storage sheath and the proximal hub.

18. The system of claim 13, wherein the first and second axial stiffnesses are approximately equal.

19. The system of claim 13, wherein the first and second axial stiffnesses are different.
